(19) United States
(12) Patent Application Publication
(10) Pub. No.: US 2015/0133647 A1
(43) Pub. Date: May 14, 2015

(54) Method for Producing Oligosaccharides and Oligosaccharide Glycosides by Fermentation

(71) Applicant: Glycom A/S, Kongens Lyngby (DK)

(72) Inventors: Pauline Peltier-Pain, Orleans (FR); Gyula Dekany, Sinnamon Park (AU); Rémy Dureau, Couzeix (FR); Christian Risinger, Rottweil (DE); Markus Hederos, Svedala (SE); Elise Champion, Toulouse (FR)

(73) Assignee: Glycom A/S, Kongens Lydgby (DK)

(21) Appl. No.: 14/406,379

(22) PCT Filed: Jun. 7, 2013
(86) PCT No.: PCT/DK2013/050182

§ 371 Date: Dec. 8, 2014

(30) Foreign Application Priority Data
Jun. 8, 2012 (DK) PA 2012 7013
Jun. 11, 2012 (DK) PA 2012 00395
Jun. 15, 2012 (DK) PA 2012 70332
Oct. 25, 2012 (DK) PA 2012 70656

(51) Int. Cl. C12P 19/44; C12P 19/46

(52) U.S. Cl. CPC C12P 19/44; C12P 19/46
USPC 536/17.9; 435/74; 536/4.1; 435/79

(57) ABSTRACT

The application discloses a method for producing anomerically protected glycosidic oligosaccharide derivatives comprising the step of culturing, in a culture medium containing an anomerically protected lactose acceptor, a genetically modified cell having a recombinant gene that encodes a glycosyl transferase that can transfer a glycosyl residue of an activated sugar nucleotide to said lactose acceptor. The application further discloses a method for producing an oligosaccharide comprising the steps of: (a) culturing, in a culture medium containing an anomerically protected lactose acceptor, a genetically modified cell having a recombinant gene that encodes a glycosyl transferase that can transfer a glycosyl residue of an activated sugar nucleotide to said lactose acceptor to produce an anomerically protected glycosidic oligosaccharide derivative, then (b) removing/deprotecting the anomeric protective group.

### FIELD OF THE INVENTION

The present invention relates to a method of making glycosides of oligosaccharides or glycosidic derivatives of oligosaccharides, particularly of human milk oligosaccharides (HMOs).

### BACKGROUND OF THE INVENTION

Human milk oligosaccharides (HMOs) have become of great interest in the past few years due to their important functions in human development. To date, the structures of at least 115 HMOs have been determined (see Urashima et al.: Milk Oligosaccharides, Nova Biomedical Books, New York, 2011, ISBN: 978-1-61122-831-1), and considerably more are probably present in human milk. The thirteen core structures identified to date, for the 115 HMOs, are listed in Table 1:

TABLE 1
Core HMO structures

| No | Core name | Core structure |
|---|---|---|
| 1 | lactose (Lac) | Galβ1-4Glc |
| 2 | lacto-N-tetraose (LNT) | Galβ1-3GlcNAcβ1-3Galβ1-4Glc |
| 3 | lacto-N-neotetraose (LNnT) | Galβ1-4GlcNAcβ1-3Galβ1-4Glc |
| 4 | lacto-N-hexaose (LNH) | Galβ1-3GlcNAcβ1-3(Galβ1-4GlcNAcβ1-6)Galβ1-4Glc |
| 5 | lacto-N-neohexaose (LNnH) | Galβ1-4GlcNAcβ1-3(Galβ1-4GlcNAcβ1-6)Galβ1-4Glc |
| 6 | para-lacto-N-hexaose (para-LNH) | Galβ1-3GlcNAcβ1-3Galβ1-4GlcNAcβ1-3Galβ1-4Glc |
| 7 | para-lacto-N-neohexaose (para- | Galβ1-4GlcNAcβ1-3Galβ1-4GlcNAcβ1-3Galβ1-4Glc |
| | LNnH) | |
| 8 | lacto-N-octaose (LNO) | Galβ1-3GlcNAcβ1-3(Galβ1-4GlcNAcβ1-3Galβ1- |
| | | 4GlcNAcβ1-6)Galβ1-4Glc |
| 9 | lacto-N-neooctaose (LNnO) | Galβ1-4GlcNAcβ1-3(Galβ1-3GlcNAcβ1-3Galβ1- |
| | | 4GlcNAcβ1-6)Galβ1-4Glc |
| 10 | iso-lacto-N-octaose (iso-LNO) | Galβ1-3GlcNAcβ1-3(Galβ1-3GlcNAcβ1-3Galβ1- |
| | | 4GlcNAcβ1-6)Galβ1-4Glc |
| 11 | para-lacto-N-octaose (para-LNO) | Galβ1-3GlcNAcβ1-3Galβ1-4GlcNAcβ1-3Galβ1- |
| | | 4GlcNAcβ1-3Galβ1-4Glc |
| 12 | lacto-N-neodecaose (LNnD) | Galβ1-3GlcNAcβ1-3[Galβ1-4GlcNAcβ1-3(Galβ1- |
| | | 4GlcNAcβ1-6)Galβ1-4GlcNAcβ1-6]Galβ1-4Glc |
| 13 | lacto-N-decaose (LND) | Galβ1-3GlcNAcβ1-3[Galβ1-3GlcNAcβ1-3(Galβ1- |
| | | 4GlcNAcβ1-6)Galβ1-4GlcNAcβ1-6]Galβ1-4Glc |

Low cost ways have been sought for making industrial quantities of as many as possible of the HMOs, so that their uses in nutritional and therapeutic formulations for infants, as well as possibly children and adults, could be discovered, developed and exploited by researchers worldwide. A few HMOs have recently been chemically or enzymatically synthesized, for example, by hydrogenating their benzyl glycoside precursors after removing other protecting groups from such precursors and then isolating (e.g. by crystallization) the HMOs (WO 2011/100979, WO 2011/100980, WO 2012/007585, WO 2012/007588, WO 2012/113405, WO 2012/127410, WO 2012/155916).

However, chemically or enzymatically synthesizing anomerically protected precursors of

HMOs like benzyl glycosides or glycosidic HMOs has required numerous complicated and costly steps, either on the donor or the acceptor side. Simpler and cheaper alternative can be the in vivo microbial production of HMO derivatives comprising glycosylation of an appropriate simple acceptor like lactose derivatives. However, the two anomerically protected lactose successfully internalized so far under fermentation conditions are allyl and propargyl lactosides, which have been supposed to be made as functionalized intermediates allowing subsequent chemical or enzymatic conjugation to another species (solid support, protein, oligonucleotide, peptide) rather than those suitable for easy or straightforward anomeric deprotection (Fort et al. Chem. Comm. 2558 (2005), EP-A-1911850). Therefore further ways have been sought for producing HMOs via their glycosides or glycosidic derivatives.

### SUMMARY OF THE INVENTION

The first aspect of this invention relates to a method for producing an oligosaccharide derivative having an aglycon R, wherein R is OR₁, which R₁ is a group removable by catalytic hydrogenolysis, or R is —SR₂, which R₂ is selected from optionally substituted alkyl, optionally substituted aryl and optionally substituted benzyl, or R is azide, or R is —NH—C(R″)═C(R′)₂, wherein each R′ independently of each other is an electron withdrawing group selected from —CN, —COOH, —COO-alkyl, —CO-alkyl, —CONH₂, —CONH-alkyl and —CON(alkyl)₂, or wherein the two R′-groups are linked together and represent —CO—(CH₂)₂₋₄—CO—and thus form with the carbon atom to which they are attached a 5-7 membered cycloalkan-1,3-dion, in which dion any of the methylene groups is optionally substituted with 1 or 2 alkyl groups, and R″ is H or alkyl,

said method comprising the step of culturing, in a culture medium containing a lactose acceptor having the aglycon R, wherein R is as defined above, a genetically modified cell having a recombinant gene that encodes an enzyme capable of modifying said lactose acceptor or one of the intermediates in the biosynthetic pathway of said oligosaccharide derivative from said lactose acceptor and that is necessary for the synthesis of said oligosaccharide derivative from said lactose acceptor.

Advantageously, said recombinant gene encodes a glycosyl transferase that can transfer a glycosyl residue of an activated sugar nucleotide to said lactose acceptor.

Further advantageously, it is provided a method for producing an oligosaccharide derivative having an aglycon R, wherein R is as defined above, using a genetically modified cell starting with an internalized lactose derivative having an aglycon R, wherein R means as above, the method comprises the steps of:

(i) obtaining a genetically modified cell, particularly a Lac Z⁻Y⁺E. coli cell, that comprises said recombinant gene,

(ii) culturing said cell in a carbon-based substrate containing culture medium in the presence of said lactose acceptor, to internalize, preferably by a mechanism of active transport, said lactose acceptor in said cell and to produce said oligosaccharide derivative by said cell.

Still advantageously, said oligosaccharide derivative having said aglycon R is separated from said culture medium, particularly after separating said cell from said culture medium.

Still further advantageously, said culturing step comprises:
(i) a first phase of exponential cell growth ensured by said carbon-based substrate, and
(ii) a second phase of cell growth limited by said carbon-based substrate which is added continuously.

An embodiment of the first aspect of the invention relates to using the method for the production of said oligosaccharide derivative having said aglycon R, wherein its oligosaccharide moiety is a human milk oligosaccharide selected from the group consisting of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT.

The second aspect of this invention relates to an oligosaccharide derivative having an aglycon R, wherein R is as defined above, particularly a human milk oligosaccharide benzyl glycoside, quite particularly a benzyl glycoside of 2′-FL, LNnT or LNT, produced by the method.

The third aspect of the invention relates to a method for producing an oligosaccharide, preferably an HMO, comprising the steps of:
a) carrying out the method according to the first aspect to obtain an oligosaccharide derivative having an aglycon R, wherein R is as defined above, then b) removing/deprotecting said aglycon R to obtain said oligosaccharide.

The fourth aspect of the invention relates to a method for producing a compound of formula

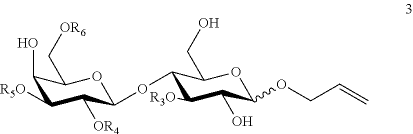

wherein R₃ is fucosyl or H, R₄ is fucosyl or H, R₅ is selected from H, sialyl, N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, R₆ is selected from H, sialyl and N-acetyl-lactosaminyl groups optionally substituted with a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, provided that at least one of the R₃, R₄, R₅ and R₆ groups is different from H, and further provided that when R₅ is sialyl then at least one of the R₃, R₄ and R₆ groups is different from H,

said method comprising the step of culturing, in a culture medium containing allyl lactoside, a genetically modified cell having a recombinant gene that encodes an enzyme capable of modifying allyl lactoside or one of the intermediates in the biosynthetic pathway of a compound of formula 3 from allyl lactoside and that is necessary for the synthesis of compound of formula 3 from allyl lactoside.

The fifth aspect of the invention relates to providing a compound of formula 3 defined above.

### DETAILED DESCRIPTION OF THE INVENTION

In accordance with this invention, it has been surprisingly discovered that exogenous carbohydrates with altered, preferably limited water solubility due to the presence of a hydrophobic aglycon thereon, wherein the aglycon can be a bulky group as well, namely exogenous carbohydrate precursors having an aglycon R, wherein R is as defined above, preferably carbohydrates containing a galactose residue, more preferably lactose derivatives can be internalized into a genetically modified cell by a transport mechanism involving permeases, allowing thus this carbohydrate precursors to be glycosylated in a genetically modified cell able to act so. It has also been found that the glycosylated products having an aglycon R, wherein R is as defined above, made by the genetically modified cell are able to be secreted to the extracellular space, and thus they can be isolated from the fermentation broth.

In this invention, the term “genetically modified cell” preferably means a cell in which at least one DNA sequence has been added to, deleted from or changed in its genome, so that the cell has a changed phenotype. This change in phenotype alters the characteristics of the genetically modified cell from that of the wild type cell. Thus, the genetically modified cell can perform at least an additional chemical transformation, when cultured or fermented, due to the added or changed DNA that encodes the expression of at least one enzyme not found in the wild type cell, or the genetically modified cell cannot perform a chemical transformation due to the deleted, added or changed DNA that encodes the expression of an enzyme found in the wild type cell. The genetically modified cell can be produced by well-known, conventional genetic engineering techniques. The genetically modified cell can be bacteria or a yeast but preferably is a bacterium. Preferred bacteria include Escherichia coli, Bacillus spp. (e.g. Bacillus subtilis), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitis, Lactobacillus spp., Lactococcus spp., Enterococcus spp., Bifidobacterium spp., Sporolactobacillus spp., Micromomospora spp., Micrococcus spp., Rhodococcus spp., Pseudomonas, particularly E. coli.

Also in this invention, the term “oligosaccharide” preferably means a sugar polymer containing at least two monosaccharide units, i.e. a di-, tri-, tetra- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g. glucose, galactose, ribose, arabinose, xylose, etc.), ketoses (e.g. fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. sialic acid). Preferably, the oligosaccharide is a HMO.

Also herein, the term “protecting group that is removable by hydrogenolysis” or “group removable by hydrogenolysis” preferably means a group having a C—O bond to the anomeric OH that can be cleaved by addition of hydrogen in the presence of catalytic amounts of palladium, Raney nickel or another appropriate metal catalyst known for use in hydrogenolysis, resulting in the regeneration of the OH group. Such protecting groups are well known to the skilled man and are discussed in Protective Groups in Organic Synthesis, PGM Wuts and TW Greene, John Wiley & Sons 2007. Suitable protecting groups include benzyl, diphenylmethyl (benzhydryl), 1-naphthylmethyl, 2-naphthylmethyl or triphenylmethyl (trityl) groups, each of which can be optionally substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). Particularly preferred protecting groups are benzyl or 1- or 2-naphthylmethyl groups optionally substituted with one or more groups selected from phenyl, alkyl or halogen. More preferably, the protecting group is selected from unsubstituted benzyl, unsubstituted 1-naphthylmethyl, unsubstituted 2-naphthylmethyl, 4-chlorobenzyl, 3-phenylbenzyl, 4-methylbenzyl and 4-nitrobenzyl.

Further herein, the term “alkyl” preferably means a linear or branched chain saturated hydrocarbon group with 1-6 carbon atoms, such as methyl, ethyl, n-propyl, i-propyl, n-butyl, i-butyl, s-butyl, t-butyl, n-hexyl, etc.; the term “aryl” preferably means a homoaromatic group such as phenyl or naphthyl; and the term “optionally substituted” preferably means a chemical group that can either carry a substituent or can be unsubstituted. More generally in connection with the terms “alkyl”, “aryl” and “benzyl”, the term “optionally substituted” preferably means that the alkyl, aryl or benzyl group can be substituted one or more times, especially 1-5 times, particularly 1-3 times with group(s) selected from alkyl (only for aryl and benzyl), hydroxy, alkoxy, carboxy, oxo, alkoxycarbonyl, alkylcarbonyl, formyl, aryl, aryloxycarbonyl, aryloxy, arylamino, arylcarbonyl, amino, mono- and dialkylamino, carbamoyl, mono- and dialkyl-aminocarbonyl, alkylcarbonylamino, cyano, alkanoyloxy, nitro, alkylthio and halogens.

The genetically modified cell used in the method of this invention comprises one or more endogenous or recombinant genes encoding one or more glycosyl transferase enzymes that are able to transfer the glycosyl residue of an activated sugar nucleotide to an internalized acceptor molecule. The gene or an equivalent DNA sequence thereof, if it is recombinant, is introduced into the cell by known techniques, using an expression vector. The origin of the heterologous nucleic acid sequence can be any animal (including human) or plant, eukaryotic cells such as those from Saccharomyces cerevisae, Saccharomyces pombe, Candida albicans and the like, prokaryotic cells such as those originated from E. coli, Bacillus subtilis, Campylobacter pylori, Helicobacter pylori, Agrobacterium tumefaciens, Staphylococcus aureus, Thermophilus aquaticus, Azorhizobium caulinodans, Rhizobium leguminosarum, Rhizobium meliloti, Neisseria gonorrhoeae and Neisseria meningitis, or virus. The glycosyl transferase enzyme/enzymes expressed by the protein(s) encoded by the gene(s) or equivalent DNA sequence(s) are preferably glucosyl transferases, galactosyl transferases, N-acetylglucosaminyl transferases, N-acetylgalactosaminyl transferases, glucuronosyl transferases, xylosyl transferases, mannosyl transferases, fucosyl transferases, sialyl transferases and the like. In a preferred embodiment, the glycosyl transferases are selected from the group consisting of β-1,3-N-acetylglucosaminyl transferase, β-1,3-galactosyl transferase, β-1,3-N-acetylgalactosaminyl transferase, β-1,3-glucuronosyl transferase, (β-1,6-N-acetylglucosaminyl transferase, β-1,4-N-acetylgalactosaminyl transferase, β-1,4-galactosyl transferase, α-1,3-galactosyl transferase, α-1,4-galactosyl transferase, α-2,3-sialyl transferase, α-2,6-sialyl transferase, α-2,8-sialyl transferase, α-1,2-fucosyl transferase, α-1,3-fucosyl transferase and α-1,4-fucosyl transferase. More preferably, the glycosyl transferases are selected from those involved in the construction of HMO core structures nos. 2-13 shown in Table 1, fucosylated and/or sialylated HMOs and glycosidic derivatives thereof, particularly those having an aglycon R, that is β-1,3-N-acetylglucosaminyl transferase, (β-1,6-N-acetylglucosaminyl transferase, β-1,3-galactosyl transferase, β-1,4-galactosyl transferase, α-2,3-sialyl transferase, α-2,6-sialyl transferase, α-1,2-fucosyl transferase, α-1,3-fucosyl transferase and/or α-1,4 fucosyl transferase. The genes encoding the above-mentioned transferases have been described in the literature.

When carrying out the method of this invention, a glycosyl transferase mediated glycosylation reaction preferably takes place in which an activated sugar nucleotide serves as donor. An activated sugar nucleotide generally has a phosphorylated glycosyl residue attached to a nucleoside, a specific glycosyl transferase enzyme accept only a specific sugar nucleotide. Thus, preferably the following activated sugar nucleotides are involved in the glycosyl transfer: UDP-Glc, UDP-Gal, UDP-GlcNAc, UDP-GalNAc, UDP-glucuronic acid, GDP-Fuc and CMP-sialic acid, particularly those selected from the group consisting of UDP-Gal, UDP-GlcNAc, GDP-Fuc and CMP-sialic acid.

In one embodiment of the method, the genetically modified cell is able to produce one or more activated sugar nucleotide mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, fructose or glucose (for a review for monosaccharide metabolism see e.g. H. H. Freeze and A. D. Elbein: Chapter 4: Glycosylation precursors, in: Essentials of Glycobiology, 2^{nd} edition (Eds. A. Varki et al.), Cold Spring Harbour Laboratory Press (2009)).The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharide for producing activated sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the cell used for genetic modification. Preferably, the synthesis of GDP-fucose or CMP-sialic acid can be accomplished using the salvage pathway, when exogenous fucose or sialic acid is also added to the culture.

According to the preferred embodiment disclosed above, the genetically modified cell is cultured in the presence of a carbon-based substrate such as glycerol, glucose, glycogene, fructose, maltose, starch, cellulose, pectin, chitin, etc. Preferably, the cell is cultured on glycerol and/or glucose and/or fructose.

It should be emphasized, that whatever way, either the de novo, or the salvage pathway taken for producing activated sugar nucleotides by the genetically modified cell is advantageous compared to in vitro versions of transfer glycosylation, as it avoids using the very expensive sugar nucleotide type donors added exogenously, hence the donors are formed by the cell in situ and the phosphatidyl nucleoside leaving groups are recycled in the cell.

The method of the invention also involves initially transporting an exogenous lactose derivative having the aglycon R, as an acceptor molecule, from the culture medium into the genetically modified cell for glycosylation where it can be glycosylated to produce the oligosaccharide derivative. The acceptor can be added exogenously in a conventional manner to the culture medium, from which it can then be transported into the cell. The internalization of the acceptor should not, of course, affect the basic and vital functions or destroy the integrity of the cell. In one embodiment the internalization can take place via a passive transport mechanism during which the exogenous acceptor diffuses passively across the plasma membrane of the cell. The flow is directed by the concentration difference in the extra- and intracellular space with respect to the acceptor molecule to be internalized, which acceptor is supposed to pass from the place of higher concentration to the zone of lower concentration tending towards equilibrium. In other embodiment the exogenous acceptor can be internalized in the cell with the aid of an active transport mechanism, during which the exogenous acceptor diffuses across the plasma membrane of the cell under the influence of a transporter protein or permease of the cell. Lactose permease (LacY) has specificity towards galactose and simple galactosyl disaccharides like lactose. The specificity towards the sugar moiety of the substrate to be internalized can be altered by mutation by means of known recombinant DNA techniques. In a preferred embodiment the internalization of the exogenous lactose derivative acceptor takes place via an active transport mechanism mediated by lactose permease.

Culturing or fermenting the genetically modified cell according to the method of this invention can be carried out in a conventional manner. When cultured, the exogenous lactose derivative acceptor is internalized into, and accumulates in, the genetically modified cell. The internalized substrate, acting as acceptor, participates in a glycosyl transferase induced glycosylation reaction, in which a glycosyl residue of an activated nucleotide donor is transferred so that the acceptor is glycosylated giving thus a trisaccharide derivative. Optionally, when more than one glycosyl transferase is expressed by the cell, additional glycosylation reactions can occur resulting in the formation of tetra- or higher oligosaccharide derivatives. Of course, the cell preferably lacks any enzyme activity which would degrade the acceptor or the oligosaccharide derivatives produced in the cell.

At the end of culturing, the oligosaccharide glycoside as product can be accumulated both in the intra- and the extracellular matrix. The product can be transported to the supernatant in a passive way, i.e. it diffuses outside across the cell membrane. The transport can be facilitated by sugar efflux transporters, proteins that promote the effluence of sugar derivatives from the cell to the supernatant. The sugar efflux transporter can be present exogenously or endogenously and is overexpressed under the conditions of the fermentation to enhance the export of the oligosaccharide derivative produced. The specificity towards the sugar moiety of the product to be secreted can be altered by mutation by means of known recombinant DNA techniques.

According to a preferred embodiment, the method also comprises the addition of an inducer to the culture medium. The role of the inducer is to promote the expression of enzymes involved in the de novo or salvage pathway and/or of permeases involved in the active transport and/or of sugar efflux transporters of the cell. Preferably, the inducer is isopropyl β-D-thiogalactoside (IPTG).

After carrying out the method of this invention, the oligosaccharide derivative having the aglycon R formed can be collected from the culture or fermentation broth in a conventional manner. The supernatant containing the oligosaccharide glycoside can be separated from the cells by centrifugation. The separated cells can be resuspended in water and subjected to heat and/or acid treatment in order to permeabilize them for releasing the oligosaccharide glycoside accumulated intracellularly. The product can be separated from the treated cell by centrifugation. The two supernatants containing the extra- and intracellular products, respectively, are combined and the products can be purified and isolated by means of standard separation, purification and isolation techniques such as gel and/or cationic ion exchange resin (H⁺ form) chromatography. Preferably, the oligosaccharide derivative is collected only from the supernatant. In this regard the concentration of the oligosaccharide derivative, particularly a trisaccharide derivative, especially a glycosidic 2′-FL derivative, in the extracellular fraction of the culture is surprisingly high using only the normal secreting mechanism of the cell.

The lactose derivative acceptors used in the method are known compounds that can be prepared by conventional methods. In this regard, the lactose acceptors having aglycon R, wherein R is OR₁, which R₁ is a group removable by catalytic hydrogenolysis, or R is —SR₂, which R₂ is selected from optionally substituted alkyl, optionally substituted aryl and optionally substituted benzyl, or R is azido, can be synthesized by treating lactose with acetic anhydride and sodium acetate followed by a Lewis acid catalysed glycosylation using R₁—OH or R₂—SH, preferably a benzyl/substituted benzyl alcohol or alkyl-, benzyl- or phenyl-SH in an organic solvent such as DCM, toluene or THF, or followed by a treatment with sodium azide. Subsequently, a Zemplen deprotection readily provides the above mentioned lactose O- and S-glycosides, or lactosyl azide, respectively. The vinylogous glycosyl amine acceptors can be synthesized by the treatment of lactose with aqueous ammonium hydrogen carbonate followed by the reaction of the resulting lactosyl amine with an activated vinyl reagent, such as an alkoxymethylenated or dialkylaminomethylenated malonic acid derivative, in the presence or absence of a base (Ortiz Mellet et al. J. Carbohydr. Chem. 12, 487 (1993); WO 2007/104311).

In accordance with this invention, an oligosaccharide derivative having an aglycon R, wherein R is as defined above, can be produced by fermenting a genetically modified cell starting with at least one internalized exogenous precursor consisting of a lactose derivative having an aglycon R, the method comprises the steps of:

(i) obtaining a Lac Z⁻Y⁺l E. coli cell that comprises at least one recombinant gene encoding an enzyme capable of modifying the exogenous precursor or one of the intermediates in the biosynthetic pathway of the oligosaccharide derivative having an aglycon R from the exogenous precursor necessary for the synthesis of the oligosaccharide derivative having an aglycon R from the exogenous precursor, and also the components for expressing the gene in the cell; and

(ii) culturing the cell on a carbon-based substrate in the presence of the exogenous precursor, under conditions inducing the internalization according to a mechanism of active transport of the exogenous precursor by the cell and the production of the oligosaccharide derivative having an aglycon R by the cell.

Preferably, the Lac Z⁻Y⁺ E. coli cell is cultured in the following way:

(a) a first phase of exponential cell growth ensured by a carbon-based substrate, and

(b) a second phase of cell growth limited by a carbon-based substrate which is added continuously.

More preferably, said enzyme capable of modifying the exogenous precursor or one of the intermediates in the biosynthetic pathway is an enzyme capable of performing a glycosylation by means of, preferably exogenous, glycosyl transferases.

Also preferably, said carbon-based substrate is selected from the group consisting of glycerol and glucose. More preferably, the carbon-based substrate added during the second phase glycerol.

Also preferably, said culturing is performed under conditions allowing the production of a culture with a high cell density.

Also preferably, said culturing further comprises a third phase of slowed cell growth obtained by continuously adding to the culture an amount of said carbon-based substrate that is less than the amount of the carbon-based substrate added in said second phase so as to increase the content of the oligosaccharide derivative having an aglycon R produced in the high cell density culture.

Also preferably, the amount of the carbon-based substrate added continuously to the cell culture during said third phase is at least 30% less than the amount of the carbon-based substrate added continuously during said second phase.

Also preferably, the method further comprises the addition of an inducer to said culture medium to induce the expression in said cell of said enzyme and/or of a protein involved in said transport. The inducer is preferably isopropyl β-D-thiogalactoside (IPTG) and the protein is lactose permease.

The exogenous lactose derivative having the aglycon R to be internalized by and glycosylated in the fermented cell can be added to the culture medium at once or continuously. If added at once, it is done at the end of the first phase of exponential cell growth. A concentrated aqueous solution of the acceptor is added to reach a concentration of not more than 15 g/I, preferably of about 3-5 g/I calculated on the volume of the culture, then the fermentation is continued by addition of the carbon-based substrate as described above. Alternatively, the continuous addition is beneficial when higher amount exogenous acceptor is intended to be used at a given volume. To avoid overflow metabolism and other side processes during the fermentation, the exogenous acceptor is dissolved in the feeding solution to be added during the second (and optionally the third) phase, therefore a continuous addition of the acceptor (with the carbon-based substrate) is realized.

Also preferably, the method is able to produce an oligosaccharide derivative having an aglycon R, wherein the oligosaccharide is a human milk oligosaccharide selected from the group consisting of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT, and R is as defined above.

Also preferably, an exogenous precursor of the lactose derivative of formula 1

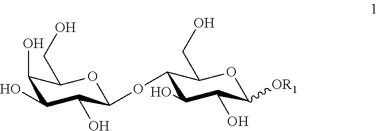

wherein R₁ is a group removable by catalytic hydrogenolysis, preferably optionally substituted benzyl, more preferably benzyl,

is used in the method to obtain an oligosaccharide having an aglycon —OR₁, wherein R₁ is defined above.

Further preferably, an exogenous precursor of the lactose derivative of formula 2

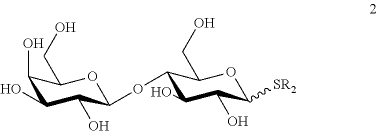

wherein R₂ is selected from optionally substituted alkyl, optionally substituted aryl and optionally substituted benzyl, preferably alkyl and phenyl,

is used in the method to obtain an oligosaccharide having an aglycon —SR₂, wherein R₂ is defined above.

Yet further preferably, lactosyl azide as an exogenous precursor is used in the method to obtain an oligosaccharide having an azido aglycon.

The method can be carried out as described in U.S. Pat. No. 7,521,212 and PCT publication WO 01/04341 A1, which are incorporated herein by reference, by adding a lactoside precursor having an aglycon R, preferably that of formula 1 or 2, or lactosyl azide, to the fermentation broth of the LacZ⁻Y⁺ E. coli, described above.

Preferably, the resulting oligosaccharide derivative obtainable by the method described above is selected from LNT, LNnT and 2′-FL having an aglycon R, fermenting a genetically modified LacZ⁻Y⁺ E. coli having genes expressing β-1,3-N-acetyl-glucosaminyl transferase and (β-1,3-galactosyl transferase for making an LNT derivative, β-1,3-N-acetyl-glucosaminyl transferase and β-1,4-galactosyl transferase for making a LNnT derivative, or α-1,2-fucosyl transferase for making a 2′-FL derivative.

The resulting oligosaccharide having the aglycon R, preferably having the aglycon —OR₁, —SR₂ or azido, more preferably having the aglycon O-benzyl/O-substituted benzyl, -S-alkyl, -S-phenyl or azido, can be isolated in a conventional manner from the aqueous fermentation broth, in which the LacZ⁻Y⁺ E. coli cell was cultured. Preferably, the aqueous fermentation broth is preferably separated (for example, by centrifugation) from the fermented E. coli, cells, filtered and then contacted with cationic and anionic ion exchange resins to remove proteins and ionic compounds. The resulting aqueous medium can then be dried (for example, by freeze drying).

If the oligosaccharide having an aglycon R, preferably benzyl/substituted benzyl glycosides thereof is to be isolated/purified by crystallization, the resulting supernatant after fermentation preferably contains no more than about 8-10 wt % and at least about 15 wt %, especially at least about 25 wt % of the glycoside. The dry, preferably protein-free glycoside powder can then be treated with a hot, preferably boiling, solvent, such as a C₁-C₆ alcohol solvent, and the resulting solution can be filtered while still hot, then kept hot to concentrate it by partial evaporation down to at least about 60-70% of its original volume and then allowed to cool somewhat. Seed crystals of the desired glycoside can then be added to the concentrated solution while it is still warm, the solution can then be allowed to cool to room temperature, and precipitated crystals of the glycoside can then be filtered from the solution.

The internalization of lactose derivatives having an aglycon R is surprising. No glycosidic lactosides have been internalized successfully in preparative scale so far except for allyl and propargyl lactosides (Fort et al. Chem. Comm. 2558 (2005), EP-A-1911850). However, the efficient transportation of compounds of formula 1 by a lactose permease unexpected due to the significant difference in bulkiness, conformation and hydrophobicity of an allyl/propargyl moiety vs a benzyl/substituted benzyl group. Moreover, the internalization of thiolactosides and their transformation by means of glycosylation in a living cell under culturing is also surprising and the present invention is the first example to show this. In addition, Fort et al. reported on the unsuccessful utilization of a lactose N-glycoside, and another lactoside having an azido group in the anomeric substituent gave poor result in a fermentation process; in this view the internalization of the lactosyl azide and its transformation by means of glycosylation in a living cell under culturing can be considered non-expected.

In accordance with the above, the second aspect of the invention relates to an oligosaccharide derivative having an aglycon R, wherein R is OR₁, which R₁ is a group removable by catalytic hydrogenolysis, or R is —SR₂, which R₂ is selected from optionally substituted alkyl, optionally substituted aryl and optionally substituted benzyl, or R is azide, or R is —NH—C(R″)═C(R′)₂, wherein each R′ independently of each other is an electron withdrawing group selected from —CN, —COOH, —COO-alkyl, —CO-alkyl, —CONH₂, —CONH-alkyl and —CON(alkyl)₂, or wherein the two R′-groups are linked together and represent —CO—(CH₂)₂₋₄—CO— and thus form with the carbon atom to which they are attached a 5-7 membered cycloalkan-1,3-dion, in which dion any of the methylene groups is optionally substituted with 1 or 2 alkyl groups, and R″ is H or alkyl, that is produced by the method according to the first aspect. In this regard, it is provided an oligosaccharide derivative defined above made by the method comprising the step of: culturing, in a culture medium containing a lactose acceptor having the aglycon R, wherein R is as defined above, a genetically modified cell having a recombinant gene that encodes an enzyme capable of modifying said lactose acceptor or one of the intermediates in the biosynthetic pathway of said oligosaccharide derivative from said lactose acceptor and that is necessary for the synthesis of said oligosaccharide derivative from said lactose acceptor.

Preferably, R is selected from —OR₁, —SR₂ and azido, wherein R₁ is a group removable by catalytic hydrogenolysis, particularly benzyl or substituted benzyl, R₂ is selected from alkyl, aryl and benzyl, particularly alkyl and phenyl.

Also preferably, the oligosaccharide derivative obtainable by the method is a HMO, particularly 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT derivatives having an aglycon R.

More preferably, the oligosaccharide derivatives obtainable by the method is selected from —O-benzyl glycoside, —O-substituted benzyl glycoside, —S-alkyl thioglycoside, —S-phenyl thioglycoside and 1-azido-1-deoxy derivative of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT, particularly —O-benzyl glycoside, —O-alkyl thioglycoside, —S-phenyl thioglycoside and 1-azido-1-deoxy derivative of 2′-FL, 3-FL, difucosyllactose, 3′-SL, 6′-SL, sialyl-fucosyl lactose, LNT and LNnT.

The advantage of making oligosaccharides having an aglycon R, preferably when R means —O-benzyl or —O-substituted benzyl, or R means —S-alkyl or —S-phenyl, or R means azido, over preparing the free oligosaccharides directly lies upon the fact, that these derivatives has limited water solubility due to the presence of the more hydrophobic group R, thus allowing the practitioner to enlarge the repertoire of e.g. chromatographic separations. For example, due to the different polarity of the glycoside derivatives vs. the free oligosaccharides a reverse phase chromatographic separation could be easily performed when water is used, as oligosaccharides having an aglycon R migrate much more slowly than the very polar compounds present in the reaction mixture, thus the polar compounds can be eluted smoothly. Oligosaccharides having an aglycon R can be then washed from the column with e.g. alcohol. Secondly, when R-group contains an aromatic moiety (e.g. phenyl), it can serve as chromophore offering the possibility of UV-detection which eases the identification of the desired objects. Thirdly, with careful selection of R-groups crystalline materials can be obtained. Crystallization or recrystallization is one of the simplest and cheapest methods to isolate a product from a reaction mixture, separate it from contaminations and obtain pure substance. Isolation or purification that uses crystallization makes the whole technological process robust and cost-effective, thus it is advantageous and attractive compared to other procedures (see above). Fourthly, removal of the anomeric protective group from a glycosidic oligosaccharide derivate obtained in the method claimed takes place under delicate conditions nearly quantitatively. For example benzyl/substituted benzyl protective groups in —OR₁ can be converted exclusively into toluene/substituted toluene under the hydrogenolysis condition and they can easily be removed even in multi ton scales from water soluble oligosaccharide products via evaporation and/or extraction processes. The compounds having R as —SR₂, wherein R₂ is optionally substituted alkyl, optionally substituted aryl or optionally substituted benzyl, can be converted into the corresponding reducing oligosaccharides in the following way: the thioglycoside is dissolved in water or a dipolar aprotic solvent containing water followed by the addition of a thiophilic activator such as mercury(II) salts, Br₂, I₂, NBS, NIS, triflic acid or triflate salts, or a mixture thereof. The activated intermediate reacts easily with the water present in the reaction milieu and a deprotected oligosaccharide can be produced. Oligosaccharides having an aglycon R, where R means azido, can be subjected to catalytic hydrogenolysis or reduced by complex metal hydrides like NaBH₄, or by PPh₃. Both types of reactions yield amine functionality at the anomeric position, the hydrolysis of which under neutral or slightly acidic pH (pH≈4-7) readily provides the deprotected oligosaccharides. Moreover, the removal of an acyclic vinylogous amine group from an oligosaccharide having an aglycon R, wherein R is —NH—C(R″)═C(R′)₂, and R′ and R″ are as defined above can be carried out by treating it with amino compounds or a halogen. Suitable solvents for this reaction include methanol, ethanol, water, acetic acid, or ethyl acetate, and mixtures thereof. Amino compounds for this reaction are the aqueous and anhydrous primary amines, such as ethylamine, propylamine and butylamine, the hydrazines, such as hydrazine hydrate and hydrazine acetate, hydroxylamine derivatives, an aqueous ammonia solution and ammonia gas. The acyclic vinylogous amine can also be cleaved with a halogen such as chlorine gas or bromine. Both types of reactions yield amine functionality at the anomeric position, the hydrolysis of which under neutral or slightly acidic pH (pH≈4-7) readily provides the deprotected oligosaccharides.

The third aspect of the invention relates to a method for synthesizing an oligosaccharide comprising the steps:

a) culturing, in a culture medium containing a lactose acceptor having an aglycon R, wherein R is OR₁, which R₁ is a group removable by catalytic hydrogenolysis, or R is —SR₂, which R₂ is selected from optionally substituted alkyl, optionally substituted aryl and optionally substituted benzyl, or R is azide, or R is —NH—C(R″)═C(R′)₂, wherein each R′ independently of each other is an electron withdrawing group selected from —CN, —COOH, —COO-alkyl, —CO-alkyl, —CONH₂, —CONH-alkyl and —CON(alkyl)₂, or wherein the two R′-groups are linked together and represent —CO—(CH₂)₂₋₄—CO— and thus form with the carbon atom to which they are attached a 5-7 membered cycloalkan-1,3-dion, in which dion any of the methylene groups is optionally substituted with 1 or 2 alkyl groups, and R″ is H or alkyl, a genetically modified cell having a recombinant gene that encodes an enzyme capable of modifying said lactose acceptor or one of the intermediates in the biosynthetic pathway of an oligosaccharide derivative having an aglycon R, wherein R is as above, from said lactose acceptor and that is necessary for the synthesis of said oligosaccharide derivative having an aglycon R from said lactose acceptor,

b) separating said oligosaccharide derivative having an aglycon R from the cell, from the culture medium or from both, and then

c) removing/deprotecting aglycon R to obtain said oligosaccharide.

In this regard, this method comprises carrying out the method disclosed in the first aspect of the present invention including the preferred and the more preferred embodiments, followed by removing/deprotecting the aglycon R from the product so-obtained by means of
catalytic hydrogenolysis, when R is —OR₁, wherein R₁ is a group removable by catalytic hydrogenolysis,
treating the product with a thiophilic activator such as mercury(II) salts, Br₂, I₂, NBS, NIS, triflic acid or triflate salts, or a mixture thereof, when R is —SR₂, wherein R₂ is optionally substituted alkyl, optionally substituted aryl or optionally substituted benzyl, followed by hydrolysis,
reducing the azido group, when R is azide, followed by hydrolysis, or
treating the product with ammonia, amino compound or halogen, R is —NH—C(R″)═C(R′)₂, and R′ and R″ are as defined above, followed by hydrolysis.

Preferably, the method according to the third aspect comprises culturing a genetically modified cell, which cell can be originated from a bacterium or yeast, more preferably a bacterium, particularly E. coli, having a gene encoding a glycosyl transferase that can transfer a glycosyl residue of an activated sugar nucleotide to a lactose acceptor having an aglycon R, wherein R is as described above, to prepare an oligosaccharide derivative having an aglycon R, and removing/deprotecting the aglycon R from them by one of the ways disclosed above.

Preferably, the method according to the third aspect comprises culturing a genetically modified cell having a glycosyl transferase selected from the group consisting of β-1,3-N-acetyl-glucosaminyl transferase, β-1,3-galactosyl transferase, β-1,3-N-acetyl-galactosaminyl transferase, β-1,3-glucuronosyl transferase, β-1,3-N-acetyl-galactosaminyl transferase, β-1,4-N-acetyl-galactosaminyl transferase, β-1,4-galactosyl transferase, α-1,3-galactosyl transferase, α-1,4-galactosyl transferase, α-2,3-sialyl transferase, α-2,6-sialyl transferase, α-2,8-sialyl transferase, α-1,2-fucosyl transferase, α-1,3-fucosyl transferase and α-1,4-fucosyl transferase, in the presence of a lactose acceptor having an aglycon R to prepare an oligosaccharide derivative having an aglycon R, and removing/deprotecting the aglycon R from them by one of the ways disclosed above.

Preferably, the method according to the third aspect comprises culturing a genetically modified cell having a glycosyl transferase, wherein the culturing is characterized by

(a) a first phase of exponential cell growth ensured by a carbon-based substrate, and

(b) a second phase of cell growth limited by a carbon-based substrate which is added continuously,

wherein the lactose acceptor having an aglycon R is added either at the end of the first phase or during the second phase, the to obtain an oligosaccharide derivative having an aglycon R, and removing/deprotecting the aglycon R from them by one of the ways disclosed above.

Preferably, the method according to the third aspect comprises the preparation of an oligosaccharide derivative having an aglycon R by a genetically modified cell, wherein the oligosaccharide is a human milk oligosaccharide, preferably a HMO selected from the group consisting of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT, and removing/deprotecting the aglycon R from them by one of the ways disclosed above to produce a human milk oligosaccharide, preferably a HMO selected from the group consisting of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT.

Preferably, the method according to the third aspect comprises the preparation of an oligosaccharide derivative having an aglycon —OR₁, —SR₂ and azido, by a genetically modified cell from a precursor:

a) of formula 1

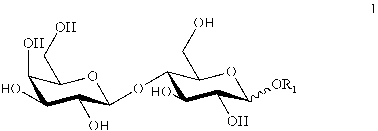

wherein R₁ is a group removable by catalytic hydrogenolysis, preferably optionally substituted benzyl, more preferably benzyl, or

b) of formula 2

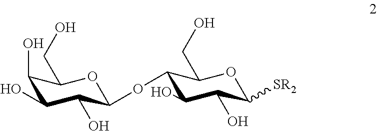

wherein R₂ is selected from optionally substituted alkyl, optionally substituted aryl and optionally substituted benzyl, preferably alkyl and phenyl, or

c) lactosyl azide,

and removing/deprotecting the aglycon
a) —OR₁ by catalytic hydrogenolysis,
b) —SR₂ by treatment with a thiophilic activator such as mercury(II) salts, Br₂, I₂, NBS, NIS, triflic acid or triflate salts, or a mixture thereof, followed by hydrolysis, or
c) azido by reducing it to amino group followed by hydrolysis,

to prepare an oligosaccharide, preferably an HMO.

More preferably, the method according to the third aspect comprises the fermentation a genetically modified cell in the presence of a precursor of formula 1 depicted above, preferably from that having an aglycon —OR₁, more preferably benzyl lactoside, to prepare an oligosaccharide derivative, preferably a LNT, LNnT and 2′-FL derivative, having an aglycon —OR₁, preferably benzyloxy, which is then subjected to catalytic hydrogenolysis to remove the R₁ group and to make an oligosaccharide, preferably LNT, LNnT and 2′-FL.

The fourth aspect of the invention relates to a method for producing a compound of formula 3

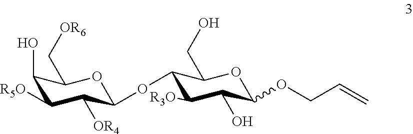

wherein R₃ is fucosyl or H, R₄ is fucosyl or H, R₅ is selected from H, sialyl, N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, R₆ is selected from H, sialyl and N-acetyl-lactosaminyl groups optionally substituted with a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, provided that at least one of the R₃, R₄, R₅ and R₆ groups is different from H, and further provided that when R₅ is sialyl then at least one of the R₃, R₄ and R₆ groups is different from H,

said method comprising the step of culturing, in a culture medium containing allyl lactoside, a genetically modified cell having a recombinant gene that encodes an enzyme capable of modifying allyl lactoside or one of the intermediates in the biosynthetic pathway of a compound of formula 3 from allyl lactoside and that is necessary for the synthesis of compound of formula 3 from allyl lactoside.

Advantageously, it is provided a method for producing a compound of formula 3 defined above using a genetically modified cell starting with an internalized allyl lactoside, the method comprises the steps of:

(i) obtaining a genetically modified cell, particularly a Lac Z⁻Y⁺ E. coli cell, that comprises said recombinant gene,

(ii) culturing said cell in a carbon-based substrate containing culture medium in the presence of allyl lactoside, to internalize, preferably by a mechanism of active transport, the allyl lactoside in said cell and to produce a compound of formula 3 by said cell.

Still advantageously, a compound of formula 3 is separated from said culture medium, particularly after separating said cell from said culture medium.

The genetically modified cell used in the method of making a compound of formula 3 comprises one or more endogenous or recombinant genes encoding one or more glycosyl transferase enzymes that are able to transfer the glycosyl residue of an activated sugar nucleotide to an internalized acceptor molecule, i.e. to allyl lactoside. The gene or an equivalent DNA sequence thereof, if it is recombinant, is introduced into the cell by known techniques, using an expression vector. The origin of the heterologous nucleic acid sequence can be any animal (including human) or plant, eukaryotic cells, prokaryotic cells or virus as described above. The glycosyl transferase enzyme/enzymes expressed by the protein(s) encoded by the gene(s) or equivalent DNA sequence(s) are as described above, with the proviso that when the genetically modified cell contains only one recombinant gene expressing a glycosyl transferase, then this glycosyl transferase is different from α-2,3-sialyl transferase.

When carrying out the method according to the fourth aspect, a glycosyl transferase mediated glycosylation reaction preferably takes place in which an activated sugar nucleotide serves as donor. An activated sugar nucleotide generally has a phosphorylated glycosyl residue attached to a nucleoside, a specific glycosyl transferase enzyme accept only a specific sugar nucleotide. Thus, preferably the following activated sugar nucleotides are involved in the glycosyl transfer: UDP-Glc, UDP-Gal, UDP-GlcNAc, UDP-GalNAc, UDP-glucuronic acid, GDP-Fuc and CMP-sialic acid, particularly those selected from the group consisting of UDP-Gal, UDP-GlcNAc and GDP-Fuc.

The genetically modified cell used in the method according to the fourth aspect is able to produce one or more activated sugar nucleotide mentioned above by a de novo pathway, or can utilize salvaged monosaccharide for producing activated sugar nucleotide (see above).

The method according to the fourth aspect also involves initially transporting the exogenous allyl lactoside, as an acceptor molecule, from the culture medium into the genetically modified cell for glycosylation where it can be glycosylated to produce an oligosaccharide derivative of formula 3. The allyl lactoside can be added exogenously in a conventional manner to the culture medium, from which it can then be transported into the cell. The internalization of the acceptor should not, of course, affect the basic and vital functions or destroy the integrity of the cell. In one embodiment the internalization can take place via a passive transport mechanism during which the exogenous acceptor diffuses passively across the plasma membrane of the cell. The flow is directed by the concentration difference in the extra- and intracellular space with respect to the acceptor molecule to be internalized, which acceptor is supposed to pass from the place of higher concentration to the zone of lower concentration tending towards equilibrium. In other embodiment the exogenous acceptor can be internalized in the cell with the aid of an active transport mechanism, during which the exogenous acceptor diffuses across the plasma membrane of the cell under the influence of a transporter protein or permease of the cell. Lactose permease (LacY) has specificity towards galactose and simple galactosyl disaccharides like lactose. The specificity towards the sugar moiety of the substrate to be internalized can be altered by mutation by means of known recombinant DNA techniques. In a preferred embodiment the internalization of the exogenous lactose derivative acceptor takes place via an active transport mechanism mediated by lactose permease.

Culturing or fermenting the genetically modified cell according to the method of the fourth aspect can be carried out in a conventional manner. When cultured, the exogenous allyl lactoside is internalized into, and accumulates in, the genetically modified cell. The internalized substrate, acting as acceptor, participates in a glycosyl transferase induced glycosylation reaction, in which a glycosyl residue of an activated nucleotide donor is transferred so that the acceptor is glycosylated giving thus a trisaccharide derivative. Optionally, when more than one glycosyl transferase is expressed by the cell, additional glycosylation reactions can occur resulting in the formation of tetra- or higher oligosaccharide derivatives. Of course, the cell preferably lacks any enzyme activity which would degrade the acceptor or the oligosaccharide derivatives produced in the cell.

At the end of culturing, the oligosaccharide glycoside of formula 3 as product can be accumulated both in the intra- and the extracellular matrix. The product can be transported to the supernatant in a passive way, i.e. it diffuses outside across the cell membrane. The transport can be facilitated by sugar efflux transporters, proteins that promote the effluence of sugar derivatives from the cell to the supernatant. The sugar efflux transporter can be present exogenously or endogenously and is overexpressed under the conditions of the fermentation to enhance the export of the oligosaccharide derivative produced. The specificity towards the sugar moiety of the product to be secreted can be altered by mutation by means of known recombinant DNA techniques.

According to a preferred embodiment, the method also comprises the addition of an inducer to the culture medium. The role of the inducer is to promote the expression of enzymes involved in the de novo or salvage pathway and/or of permeases involved in the active transport and/or of sugar efflux transporters of the cell. Preferably, the inducer is isopropyl β-D-thiogalactoside (IPTG).

After carrying out the fermentation, the oligosaccharide derivative of formula 3 formed can be collected from the culture or fermentation broth in a conventional manner. The supernatant containing the product can be separated from the cells by centrifugation. The separated cells can be resuspended in water and subjected to heat and/or acid treatment in order to permeabilize them for releasing the oligosaccharide glycoside accumulated intracellularly. The product can be separated from the treated cell by centrifugation. The two supernatants containing the extra- and intracellular products, respectively, are combined and the products can be purified and isolated by means of standard separation, purification and isolation techniques such as gel and/or cationic ion exchange resin (H⁺ form) chromatography. Preferably, the oligosaccharide derivative is collected only from the supernatant.

In accordance with the fourth aspect, a compound of formula 3 can be produced by fermenting a genetically modified cell starting with an internalized exogenous allyl lactoside, the method comprising the steps of:

(i) obtaining a Lac Z⁻Y⁺ E. coli cell that comprises at least one recombinant gene encoding an enzyme capable of modifying the exogenous precursor or one of the intermediates in the biosynthetic pathway of the oligosaccharide derivative of formula 3 from the exogenous allyl lactoside necessary for the synthesis of the oligosaccharide derivative of formula 3 from the exogenous allyl lactoside, and also the components for expressing the gene in the cell; and

(ii) culturing the cell on a carbon-based substrate in the presence of the exogenous allyl lactoside, under conditions inducing the internalization according to a mechanism of active transport of the allyl lactoside by the cell and the production of the oligosaccharide derivative of formula 3 by the cell.

Preferably, the Lac Z⁻Y⁺ E. coli cell is cultured in the following way:

(a) a first phase of exponential cell growth ensured by a carbon-based substrate, and

(b) a second phase of cell growth limited by a carbon-based substrate which is added continuously.

More preferably, said enzyme capable of modifying the exogenous allyl lactoside or one of the intermediates in the biosynthetic pathway is an enzyme capable of performing a glycosylation by means of, preferably exogenous, glycosyl transferases.

Also preferably, said carbon-based substrate is selected from the group consisting of glycerol and glucose. More preferably, the carbon-based substrate added during the second phase glycerol.

Also preferably, said culturing is performed under conditions allowing the production of a culture with a high cell density.

Also preferably, said culturing further comprises a third phase of slowed cell growth obtained by continuously adding to the culture an amount of said carbon-based substrate that is less than the amount of the carbon-based substrate added in said second phase so as to increase the content of the oligosaccharide derivative of formula 3 produced in the high cell density culture.

Also preferably, the amount of the carbon-based substrate added continuously to the cell culture during said third phase is at least 30% less than the amount of the carbon-based substrate added continuously during said second phase.

Also preferably, the method further comprises the addition of an inducer to said culture medium to induce the expression in said cell of said enzyme and/or of a protein involved in said transport. The inducer is preferably isopropyl β-D-thiogalactoside (IPTG) and the protein is lactose permease.

The exogenous allyl lactoside to be internalized by and glycosylated in the fermented cell can be added to the culture medium at once or continuously. If added at once, it is done at the end of the first phase of exponential cell growth. A concentrated aqueous solution of the acceptor is added to reach a concentration of not more than 15 g/l, preferably of about 3-5 g/l calculated on the volume of the culture, then the fermentation is continued by addition of the carbon-based substrate as described above. Alternatively, the continuous addition is beneficial when higher amount exogenous acceptor is intended to be used at a given volume. To avoid overflow metabolism and other side processes during the fermentation, the exogenous acceptor is dissolved in the feeding solution to be added during the second (and optionally the third) phase, therefore a continuous addition of the acceptor (with the carbon-based substrate) is realized.

Also preferably, the method is able to produce an oligosaccharide derivative of formula 3 characterized by formula 3a, 3b or 3c

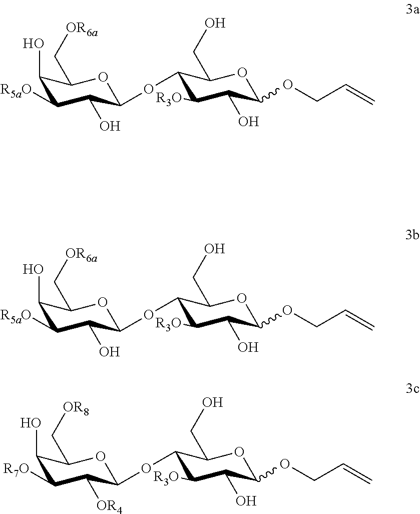

wherein R₃ and R₄ are as defined above,

R₅ₐ is an N-acetyl-lactosaminyl group optionally substituted with a glycosyl residue comprising one N-acetyl-lactosaminyl and/or one lacto-N-biosyl group; each of the N-acetyl-lactosaminyl and lacto-N-biosyl groups can be substituted with one or more sialyl and/or fucosyl residue,

R₆ₐ is H or an N-acetyl-lactosaminyl group optionally substituted with a lacto-N-biosyl group; each of the N-acetyl-lactosaminyl and lacto-N-biosyl groups can be substituted with one or more sialyl and/or fucosyl residue,

R_{5b} is a lacto-N-biosyl group optionally substituted with one or more sialyl and/or fucosyl residue(s),

R_{6b} is H or an N-acetyl-lactosaminyl group optionally substituted with one or two N-acetyl-lactosaminyl and/or one lacto-N-biosyl groups; each of the N-acetyl-lactosaminyl and lacto-N-biosyl groups can be substituted with one or more sialyl and/or fucosyl residues,

R₇ and R₈ are, independently, H or sialyl,

provided that at least one of R₃, R₄, R₇ and R₈ is not H, and further provided that when R₇ is sialyl then at least one of R₃, R₄ and R₈ is not H.

More preferably, the compounds according to formulae 3a or 3b obtainable by the method of the fourth aspect are characterized in that:
the N-acetyl-lactosaminyl group in the glycosyl residue of R₅ₐ is attached to another N-acetyl-lactosaminyl group with a 1-3 interglycosidic linkage,
the lacto-N-biosyl group in the glycosyl residue of R₅ₐ is attached to the N-acetyl-lactosaminyl group with a 1-3 interglycosidic linkage,
the lacto-N-biosyl group in the glycosyl residue of R₆ₐ is attached to the N-acetyl-lactosaminyl group with a 1-3 interglycosidic linkage,
the N-acetyl-lactosaminyl group in the glycosyl residue of R_{6b} is attached to another N-acetyl-lactosaminyl group with a 1-3 or a 1-6 interglycosidic linkage,
the lacto-N-biosyl group in the glycosyl residue of R_{6b} is attached to the N-acetyl-lactosaminyl group with a 1-3 interglycosidic linkage.

Also more preferably, a compound of formula 3a obtainable by the method of the fourth aspect is allyl glycoside of lacto-N-neotetraose, para-lacto-N-hexaose, para-lacto-N-neohexaose, lacto-N-neohexaose, para-lacto-N-octaose or lacto-N-neooctaose optionally substituted with one or more sialyl and/or fucosyl residue, and a compound of formula 3b obtainable by the method of the fourth aspect is allyl glycoside of lacto-N-tetraose, lacto-N-hexaose, lacto-N-octaose, iso-lacto-N-octaose, lacto-N-decaose or lacto-N-neodecaose optionally substituted with one or more sialyl and/or fucosyl residue.

Preferably, the compounds of formula 3a or 3b obtainable by the method of the fourth aspect are characterized in that:

the fucosyl residue attached to the N-acetyl-lactosaminyl and/or the lacto-N-biosyl group is linked to
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage,

the sialyl residue attached to the N-acetyl-lactosaminyl and/or the lacto-N-biosyl group is linked to
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage.

According to a further preferred aspect, a compound according to subformulae 3a, 3b or 3c obtainable by the method of the fourth aspect may be selected from the group of: allyl glycoside of 2′-fucosyllactose, 3-fucosyllactose, 2′,3-difucosyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II, or salts thereof. The glycosides may be alpha or beta-anomers, but preferably a beta-anomer.

The method according to the fourth aspect can be carried out as described in WO 01/04341 A1 and Fort et al. Chem. Comm. 2558 (2005), which are incorporated herein by reference, by adding allyl lactoside to the fermentation broth of the LacZ⁻Y⁺ E. coli, described above.

Preferably, the resulting oligosaccharide derivative obtainable by the method described above is selected from LNT, LNnT and 2′-FL allyl glycoside, fermenting a genetically modified LacZ⁻Y⁺ E. coli having genes expressing β-1,3-N-acetyl-glucosaminyl transferase and (β-1,3-galactosyl transferase for making LNT allyl glycoside, β-1,3-N-acetyl-glucosaminyl transferase and β-1,4-galactosyl transferase for making LNnT allyl glycoside, or α-1,2-fucosyl transferase for making 2′-FL allyl glycoside.

The fifth aspect of the invention provides a compound of formula 3

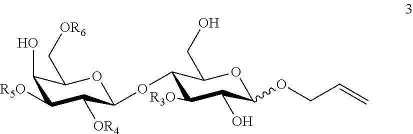

wherein R₃ is fucosyl or H, R₄ is fucosyl or H, R₅ is selected from H, sialyl, N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, R₆ is selected from H, sialyl and N-acetyl-lactosaminyl groups optionally substituted with a glycosyl residue comprising one or more N-acetyl-lactosaminyl and/or one or more lacto-N-biosyl groups; each of the N-acetyl-lactosaminyl and lacto-N-biosyl groups can be substituted with one or more sialyl and/or fucosyl residue, provided that at least one of the R₃, R₄, R₅ and R₆ groups is different from H, and further provided that when R₅ is sialyl then at least one of the R₃, R₄ and R₆ groups is different from H.

Preferably, an oligosaccharide derivative of formula 3 is characterized by formula 3a, 3b or 3c

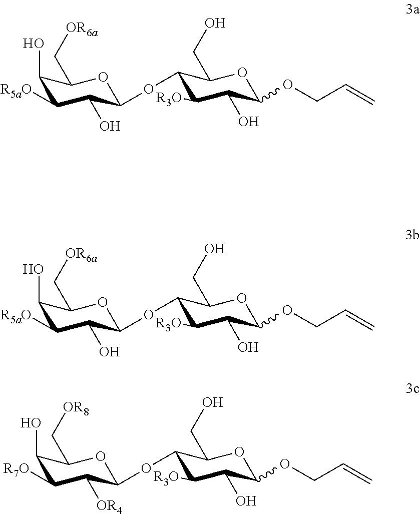

wherein R₃ and R₄ are as defined above,

R₅ₐ is an N-acetyl-lactosaminyl group optionally substituted with a glycosyl residue comprising one N-acetyl-lactosaminyl and/or one lacto-N-biosyl group; each of the N-acetyl-lactosaminyl and lacto-N-biosyl groups can be substituted with one or more sialyl and/or fucosyl residue,

R₆ₐ is H or an N-acetyl-lactosaminyl group optionally substituted with a lacto-N-biosyl group; each of the N-acetyl-lactosaminyl and lacto-N-biosyl groups can be substituted with one or more sialyl and/or fucosyl residue,

R_{5b} is a lacto-N-biosyl group optionally substituted with one or more sialyl and/or fucosyl residue(s),

R_{6b} is H or an N-acetyl-lactosaminyl group optionally substituted with one or two N-acetyl-lactosaminyl and/or one lacto-N-biosyl groups; each of the N-acetyl-lactosaminyl and lacto-N-biosyl groups can be substituted with one or more sialyl and/or fucosyl residues,

R₇ and R₈ are, independently, H or sialyl,

provided that at least one of R₃, R₄, R₇ and R₈ is not H, and further provided that when R₇ is sialyl then at least one of R₃, R₄ and R₈ is not H.

More preferably, the compounds according to formulae 3a or 3b are characterized in that:
the N-acetyl-lactosaminyl group in the glycosyl residue of R₅ₐ is attached to another N-acetyl-lactosaminyl group with a 1-3 interglycosidic linkage,
the lacto-N-biosyl group in the glycosyl residue of R₅ₐ is attached to the N-acetyl-lactosaminyl group with a 1-3 interglycosidic linkage,
the lacto-N-biosyl group in the glycosyl residue of R₆ₐ is attached to the N-acetyl-lactosaminyl group with a 1-3 interglycosidic linkage,
the N-acetyl-lactosaminyl group in the glycosyl residue of R_{6b} is attached to another N-acetyl-lactosaminyl group with a 1-3 or a 1-6 interglycosidic linkage,
the lacto-N-biosyl group in the glycosyl residue of R_{6b} is attached to the N-acetyl-lactosaminyl group with a 1-3 interglycosidic linkage.

Also more preferably, a compound of formula 3a is allyl glycoside of lacto-N-neotetraose, para-lacto-N-hexaose, para-lacto-N-neohexaose, lacto-N-neohexaose, para-lacto-N-octaose or lacto-N-neooctaose optionally substituted with one or more sialyl and/or fucosyl residue, and a compound of formula 3b is allyl glycoside of lacto-N-tetraose, lacto-N-hexaose, lacto-N-octaose, iso-lacto-N-octaose, lacto-N-decaose or lacto-N-neodecaose optionally substituted with one or more sialyl and/or fucosyl residue.

Preferably, the compounds of formula 3a or 3b are characterized in that:
the fucosyl residue attached to the N-acetyl-lactosaminyl and/or the lacto-N-biosyl group is linked to
   the galactose of the lacto-N-biosyl group with 1-2 interglycosidic linkage and/or
   the N-acetyl-glucosamine of the lacto-N-biosyl group with 1-4 interglycosidic linkage and/or
   the N-acetyl-glucosamine of the N-acetyl-lactosaminyl group with 1-3 interglycosidic linkage,
the sialyl residue attached to the N-acetyl-lactosaminyl and/or the lacto-N-biosyl group is linked to
   the galactose of the lacto-N-biosyl group with 2-3 interglycosidic linkage and/or
   the N-acetyl-glucosamine of the lacto-N-biosyl group with 2-6 interglycosidic linkage and/or
   the galactose of the N-acetyl-lactosaminyl group with 2-6 interglycosidic linkage.

According to a further preferred aspect, a compound according to subformulae 3a, 3b or 3c may be selected from the group of: allyl glycoside of 2′-fucosyllactose, 3-fucosyllactose, 2′, 3-difucosyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II, or salts thereof. The glycosides may be alpha or beta-anomers, but preferably a beta-anomer.

Particularly preferably, a compound of formula 3 is selected from LNT, LNnT and 2′-FL allyl glycoside.

A compound of formula 3 is a useful functionalized intermediate. The double bond can be used in e.g. cycloaddition reaction to bind a compound of formula 3 to other species. The chemical transformation of the double bond to aldehyde or amine also allows subsequent chemical or enzymatic conjugation to another species (solid support, protein, oligonucleotide, peptide).

Other features of the invention will become apparent in view of the following exemplary embodiments which are illustrative but not limiting of the invention.

### EXAMPLES

Bacterial strains and inoculum preparation:

Engineered E. coli used in Examples 1 to 7 was constructed from E. coli K strain in accordance with WO 01/04341 and Drouillard et al. Angew. Chem. Int. Ed. Eng. 45, 1778 (2006), by deleting genes that are liable to degrade the acceptor, the oligosaccharide product and its metabolic intermediates, inter alia the lacZ, lacA and wcaJ genes, maintaining manB, manC, gmd and wcaG genes involved in the GDP-fucose biosynthesis, and inserting H. pylori futC gene for α-1,2-fucosyl transferase.

Engineered E. coli used in Example 8 was constructed from E. coli K strain JM109 in accordance with WO 01/04341, Dumon et al. Glycoconj. J. 18, 465 (2001) and Priem et al. Glycobiology 12, 235 (2002), by deleting genes that are liable to degrade the acceptor, the oligosaccharide product and its metabolic intermediates, inter alia the lacZ, lacA and wcaJ genes, maintaining genes involved in the UDP-GlcNAc and UDP-Gal biosynthesis, and inserting N. meningitidis IgtA gene for β-1,3-N-acetylglucosaminyl transfearse and N. meningitidis IgtB gene for β-1-4-galactosyl transferase.

Engineered E. coli used in Example 9 was constructed from E. coli K strain in accordance with WO 01/04341 and M. Randriantsoa: Synthése microbiologique des antigenes glucidiques des groupes sanguins, Thése de Doctorat soutenue le 30 Sep. 2008 a I′ Université Joseph Fourier, Grenoble, pp 64-66, by deleting genes that are liable to degrade the acceptor, the oligosaccharide product and its metabolic intermediates, inter alia the lacZ, lacA and wcaJ genes, maintaining genes involved in the UDP-GlcNAc and UDP-Gal biosynthesis, and inserting N. meningitidis IgtA gene for β-1,3-N-acetylglucosaminyl transfearse and H. pylori galTK gene for β-1-3-galactosyl transferase.

General Fermentation Procedure:

The culture was carried out in a 2 l fermenter (except if noted otherwise) containing 1.5 l of mineral culture medium (Samain et al. J. Biotechnol. 72, 33 (1999)). The temperature was kept at 33° C. and the pH regulated at 6.8 with 28% NH₄OH. The inoculum (1% of the volume of the basal medium) consisted in a LB medium and the culture of the producing strain. The exponential growth phase started with the inoculation and stopped until exhaustion of the carbon source (glucose 17.5 g/l) initially added to the medium. The acceptor (various amount, given in the examples) and the inducer (isopropyl thio-β-D-galactopyranoside, IPTG, 1-2 ml of a 50 mg/ml solution) was added at the end of the exponential phase. Then a fed-batch was realized, using a 500 g/l aqueous glycerol solution, with a high substrate feeding rate of 4.5 g/h of glycerol for 1 l of culture for 5-6 hours followed by a lower glycerol feeding rate of 3 g/h for 1 l culture for a time indicated in the examples.

Purification:

At the end of the fermentation, the culture was centrifuged for 25-40 min at 4500-6000 rpm at 20-25° C. The supernatant was kept and acidify to pH 3 using a H⁺ form resin. This resulted in the precipitation of the proteins. The resin was recovered by decantation and precipitated proteins removed by centrifugation for 25-40 min at 4500-6000 rpm at 20-25 PC. The supernatant was passed through a H⁺ form ion-exchange resin column and immediately neutralized by passing through a free base form anion exchange resin column. The compounds were eluted with water or aqueous ethanol, the flow rate was about 20 ml/min and the final pH was 6.0. The fractions containing the product were collected, concentrated and freeze-dried/crystallized/precipitated.

General LC-MS Conditions:

Instrument: Bruker microQTof II MS coupled with Dionex Ultimate 3000 UHPLC

Ionization: ESI negative

Dry temperature: 200° C.

Mode: LC-MS, 1:1 split of flow

Calibration: with Na-format cluster solution

### EXAMPLE 1

### 1-O-Benzyl-β-2′-FL

The fermentation was carried out using benzyl β-lactoside (Matsuoka et al. Carbohydr. Polymers 69, 326 (2007), 7.5 g dissolved in about 25 ml of water) which was added at once at the end of the exponential phase. The second feeding phase lasted 20 hours. After resin purification (eluent: 50% ethanol) 1.7 g of product was collected. When the elution was performed using 50% ethanol gradually changed to water, 4.6 g of product was obtained. The product was identified by MS analysis and NMR which was consistent with that reported in WO 2012/007585.

### EXAMPLE 2

### 1-O-Benzyl-β-2′-FL

The fermentation was carried out using benzyl β-lactoside (62 g) that was added to the glycerol feeding solution and thus continuously added to the fermentation broth during the fermentation which lasted 66 hours altogether. After usual work-up 71 g of product could be isolated.

### EXAMPLE 3

### 1-Azido-1-deoxy-β-2′-FL

The fermentation was carried out using β-lactosyl azide (Zhang et al. J. Carbohydr. Chem. 18, 1009 (1999), 5.0 g dissolved in about 25 ml of water) which was added at once at the end of the exponential phase. The second feeding phase lasted 20 hours. After usual work-up 3.6 g of product could be isolated (identified by MS analysis and NMR which was consistent with that reported by Li et al. Biochemistry 47, 378 (2008)).

### EXAMPLE 4

### 1-Azido-1-deoxy-β-2′-FL

The fermentation was carried out using β-lactosyl azide (60 g) that was added to the glycerol feeding solution and thus continuously added to the fermentation broth during the fermentation which lasted 60 hours altogether. After usual work-up 51 g of product could be isolated.

### EXAMPLE 5

### 1-deoxy-1-thiophenyl -β-2′-FL

The fermentation was carried out in a 1 l fermenter containing 0.75 l of mineral culture medium using phenyl 1-thio-β-lactoside (Guilbert et al. Tetrahedron: Asymmetry 5, 2163 (1994), 10.0 g dissolved in about 50 ml of water) which was added at once at the end of the exponential phase. The second feeding phase lasted 30 hours. After usual work-up 8.2 g of product could be isolated. LC-MS: 579.1858 Da [M-H]⁻, ¹H NMR (300 MHz, D₂O) δ: 7.58-7.32 (m, 5H, Hₐᵣₒₘ), 5.27 (d, J=3.2 Hz, 1H, H-1″), 4.73 (d, J=9.8 Hz, 1H), 4.47 (d, J=7.6 HZ, 1H), 4.16 (d, J=6.6 Hz, 1H), 3.97-3.33 (m, 14H), 1.16 (d, J=6.5 Hz, 3H, H-6″). ¹³C NMR (75 MHz, D₂O) δ: 131.3, 131.3, 128.9, 128.9, 127.7 (Cₐᵣₒₘ), 99.7 and 98.8 (C-1′ and C-1″), 86.8 (C-1), 78.8, 75.8, 75.2, 74.9, 74.7, 73.0, 71.1, 71.0, 69.1, 68.6, 67.7, 66.4, 60.6, 59.7, 14.8 (C-6″).

### EXAMPLE 6

### 1-deoxy-1-thiomethyl-β-2′-FL

The fermentation was carried out using methyl 1-thio-β-lactoside (Leontein et al. Carbohydr. Res. 144, 231 (1985), 4.8 g dissolved in about 25 ml of water) which was added at once at the end of the exponential phase. The second feeding phase lasted 30 hours. After usual work-up 5.7 g of product could be isolated. LC-MS: 517.1837 Da [M-H]⁻, ¹H NMR (300 MHz, D₂O) δ: 5.28 (s, 1H, H-1″), 4.54-4.37 (m, 2H), 4.20 (q, J=6.5 Hz, 1H), 4.01-3.35 (m, 15H), 2.19 (d, J=1.5 Hz, 3H, SMe), 1.25-1.16 (m, 3H, H-6″). ¹³C NMR (75 MHz, D₂O) δ: 99.7 and 98.8 (C-1′ and C-1″), 85.0 (C-1), 78.8, 75.8, 75.1, 74.7, 73.1, 71.2, 70.9, 69.1, 68.6, 67.6, 66.4, 60.6, 59.8, 14.8 (C-6″), 10.9 (SMe).

### EXAMPLE 7

### 1-O-Allyl-β-2′-FL

The fermentation was carried out using allyl β-lactoside (prepared from allyl heptaacetyl-β-lactoside [Mereyala et al. Carbohydr. Res. 307, 351 (1998)] by Zemplén deacetylation, 62 g) that was added to the glycerol feeding solution and thus continuously added to the fermentation broth during the fermentation which lasted 60 hours altogether. After usual work-up 54 g of product could be isolated. LC-MS: 527.1994 Da [M-H]^{-,} ¹H NMR (300 MHz, D₂O) δ: 6.01-5.86 (m, 1H), 5.39-5.21 (m, 3H), 4.46 (t, J=7.9 Hz, 2H), 4.41-4.30 (m, 1H), 4.23-4.13 (m, 2H), 3.97-3.58 (m, 12H), 3.58-3.50 (m, 1H), 3.45-3.35 (m, 1H), 3.34-3.26 (m, 1H), 1.18 (d, J=6.5 Hz, 3H, H-6″). ¹³C NMR (75 MHz, D₂O) δ: 133.3 (CH═CH₂), 118.9 (═CH₂), 101.3, 100.4 and 99.5 (C-1, C-1′ and C-1″), 76.4, 76.0, 75.4, 75.3, 74.4, 73.7, 73.0, 71.8, 70.8, 69.7, 69.2, 68.3, 67.0, 61.2, 60.3, 15.4 (C-6″).

### EXAMPLE 8

### 1-O-Benzyl-β-LNnT

The fermentation was carried out using benzyl β-lactoside (20 g) that was added to the glycerol feeding solution and thus continuously added to the fermentation broth during the fermentation which lasted 36 hours altogether. After usual work-up 18 g of product could be isolated which was identical to the sample prepared according to WO 2011/100980.

### EXAMPLE 9

### 1-O-Benzyl-β-LNT

The fermentation was carried out using benzyl β-lactoside (40 g) that was added to the glycerol feeding solution and thus continuously added to the fermentation broth during the fermentation which lasted 36 hours altogether. After usual work-up 24 g of product could be isolated. ¹H-NMR (D₂O, 400 MHz) δ: 2.03 (s, 3H, CH₃CONH), 3.35 (dd, 1H, J=8.1 8.5 Hz, H-2), 3.49 (m, 1H, H-5′′), 3.53 (m, H-2′′′ ), 3.65 (m, 1H, H-3′′′), 3.57 (dd, 1H, J=8.1 9.0 Hz, H-4′′), 3.58 (m, 1H, H-5), 3.59 (dd, 1H, J=7.7 10.0 Hz, H-2′), 3.62 (m, 1H, H-3), 3.63 (m, 1H, H-4), 3.71 (m, 1H, H-5′), 3.71 (m, 1H, H-5′′′), 3.73 (dd, 1H, J=3.3 10.0 Hz, H-3′), 3.76 (m, 2H, H-6ab′′′), 3.76 (m, 2H, H-6ab′), 3.80 (m, 1H, H-6a′′), 3.80 (dd, 1H, J=5.0 12.2 Hz, H-6a), 3.82 (dd, 1H, J=8.1 10.5 Hz, H-3′′), 3.90 (m, 1H, H-6b′′), 3.90 (dd, 1H, J=8.4 10.5 Hz, H-2′′), 3.92 (d, 1H, J=3.3 Hz, H-4′′′), 3.98 (dd, 1H, J=1.6 12.2 Hz, H-6b), 4.15 (d, 1H, J=3.3 Hz, H-41, 4.44 (d, 1H, J=7.7 Hz, H-1′), 4.45 (d, 1H, J=7.7 Hz, H-1′′′), 4.56 (d, 1H, J=8.1 Hz, H-1), 4.73 (d, 1H, J=8.4 Hz, H-1′′), 4.76 (d, 1H, J=11.7 Hz, CH₂Ph), 4.94 (d, 1H, J=11.7 Hz, CH₂Ph), 7.40-7.50 (m, 5H, Ph). ¹³C-NMR (D₂O, 100 MHz) δ: 24.9 (CH₃CONH), 57.4 (C-2′′), 62.8 (C-6), 63.2 (C-6′′), 63.7 (C-6′′′), 63.7 (C-6′), 71.0 (C-4′), 71.2 (C-4′′′), 71.3 (C-4′′), 72.7 (C-2′), 73.4 (C-2′′′), 74.2 (CH₂Ph), 75.2 (C-3′′′), 75.5 (C-2), 77.1 (C-3), 77.5 (C-5′), 77.6 (C-5′′′), 77.9 (C-5), 78.0 (C-5′′), 81.1 (C-4), 84.7 (C-3′), 84.8 (C-3′′), 103.7 (C-1), 105.3 (C-1′′), 105.6 (C-1′), 106.2 (C-1′′′), 131.1 (Ph), 131.4 (2C, Ph), 131.5 (2C, Ph), 139.2 (Ph), 177.7 (CH₃CONH).

1. A method for producing an oligosaccharide derivative having an aglycon R, wherein R is OR₁, which R₁ is a group removable by catalytic hydrogenolysis, or R is —SR₂, which R₂ is selected from optionally substituted alkyl, optionally substituted aryl and optionally substituted benzyl, or R is azide, or R is —NH—C(R″)═C(R′)₂, wherein each R′ independently of each other is an electron withdrawing group selected from —CN, —COOH, —COO-alkyl, —CO-alkyl, —CONH₂, —CONH-alkyl and —CON(alkyl)₂, or wherein the two R′-groups are linked together and represent —CO—(CH₂)₂₋₄—CO— and thus form with the carbon atom to which they are attached a 5-7 membered cycloalkan-1,3-dion, in which dion any of the methylene groups is optionally substituted with 1 or 2 alkyl groups, and R″ is H or alkyl, said method comprising the step of culturing, in a culture medium containing a lactose acceptor having the aglycon R, wherein R is as defined above, a genetically modified cell having a recombinant gene that encodes an enzyme capable of modifying said lactose acceptor or one of the intermediates in the biosynthetic pathway of said oligosaccharide derivative from said lactose acceptor and that is necessary for the synthesis of said oligosaccharide derivative from said lactose acceptor.

2. The method according to claim 1 comprising the steps of:
(i) obtaining said genetically modified cell, and
(ii) culturing said cell in a carbon-based substrate containing culture medium in the presence of said lactose acceptor to internalize it in said cell and to produce said oligosaccharide derivative by said cell.

3. The method according to claim 1 further comprising the step of separating said oligosaccharide derivative from said cell, from said culture medium or from both.

4. The method according to claim 1, wherein said encoded enzyme is an enzyme capable of performing a glycosylation, chosen from glycosyl transferases, by transferring a glycosyl residue of an activated sugar nucleotide to the lactose acceptor having an aglycon R.

5. The method according to and claim 1, wherein said cell is a bacterium or yeast.

6. The method according to claim 1, wherein said enzyme is a glycosyl transferase selected from the group consisting of β-1,3-N-acetyl-glucosaminyl transferase, β-1,3-galactosyl transferase, β-1,3-N-acetyl-galactosaminyl transferase, β-1,3-glucuronosyl transferase, β-1,3-N-acetyl-galactosaminyl transferase, β-1,4-N-acetyl-galactosaminyl transferase, β-1,4-galactosyl transferase, α-1,3-galactosyl transferase, α-1,4-galactosyl transferase, α-2,3-sialyl transferase, α-2,6-sialyl transferase, α-2,8-sialyl transferase, α-1,2-fucosyl transferase, α-1,3-fucosyl transferase and α-1,4-fucosyl transferase.

7. The method according to claim 1, wherein said culturing comprises:
(a) a first phase of exponential cell growth ensured by a carbon-based substrate, and
(b) a second phase of cell growth limited by a carbon-based substrate which is added continuously.

8. The method according to claim 7, wherein said carbon-based substrate is selected from the group consisting of glycerol and glucose.

9. The method according to claim 1, wherein said lactose acceptor is internalized by a protein assisted regulation according to an active transport mechanism.

10. The method according to claim 1, further comprising the addition of an inducer, to said culture medium to induce the expression in said cell of said enzyme and/or of a protein involved in the active transport.

11. The method according to claim 1 for the production of an oligosaccharide derivative having an aglycon R, wherein the oligosaccharide is a human milk oligosaccharide selected from the group consisting of 2′-FL, 3-FL, difucosyllactose, 3′-SL, 6′-SL, sialyl-fucosyl lactose, LNT, LNnT, sialylated and/or fucosylated LNT and sialylated and/or fucosylated LNnT, and R is as defined above.

12. The method according to claim 1 for the production of an oligosaccharide derivative having an aglycon:
a) —OR₁, wherein R₁ is a group removable by catalytic hydrogenolysis, using the lactose derivative of formula 1 as acceptor 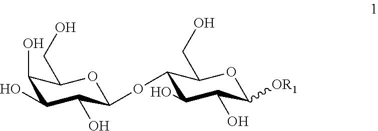
wherein R₁ is as defined above, or
b) —SR₂, wherein R₂ is selected from optionally substituted alkyl, optionally substituted aryl and optionally substituted benzyl, using of the lactose derivative of formula 2 as acceptor 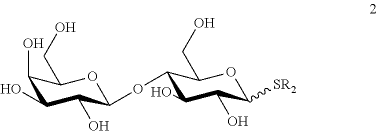
wherein R₂ is as defined above, or
c) —N₃, using lactosyl azide as acceptor.

13. The method according to claim 12, wherein the oligosaccharide derivative is that of LNT, LNnT or 2′-FL, and further comprising the addition of an inducer to said culture medium to induce the expression in said cell of said enzyme and/or of a protein involved in said transport-wherein:
said cell is a bacterium of LacZ⁻Y⁺ genotype;
said enzymes are β-1,3-N-acetyl-glucosaminyl transferase and β-1,3-galactosyl transferase for the LNT derivative, β-1,3-N-acetyl-glucosaminyl transferase and β-1,4-galactosyl transferase for the LNnT derivative, or α-1,2-fucosyl transferase for the 2′-FL derivative;
said inducer is isopropyl β-D-thiogalactoside (IPTG).

14. A method for producing an oligosaccharide comprising the steps:
a) producing an oligosaccharide derivative having an aglycon R according to claim 3, then
b) deprotecting/removing the aglycon R from the compound obtained in step a) to get the oligosaccharide.

15. The method according to claim 14, wherein
the oligosaccharide is a HMO,
step a) comprises the method according claim 3 to prepare a HMO having an aglycon —OR₁ from a precursor of formula 1 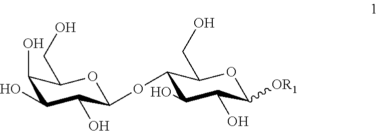
wherein R₁ is a group removable by catalytic hydrogenolysis, and
step b) is a catalytic hydrogenolysis.

16. The method according to claim 15, wherein the HMO is selected from LNT, LNnT and 2′-FL, and R₁ is benzyl.

17. An oligosaccharide derivative having an aglycon R, wherein R is as defined in claim 1, made by the method according to claim 1.

18. The method according to claim 5, wherein said cell is a bacterium of E. coli type.

19. The method according to claim 10, wherein said inducer is isopropyl β-D-thiogalactoside (IPTG), and said protein is a lactose permease.

20. The method according to claim 12, wherein R₁ is optionally substituted benzyl, and R₂ is selected from alkyl and phenyl.
